(19) United States
(12) Patent Application Publication
(10) Pub. No.: US 2008/0009466 A1
(43) Pub. Date: Jan. 10, 2008

(54) Crystalline forms of ibandronic acid and processes for preparation thereof

(76) Inventors: Sharon Avhar-Maydan, Givataym (IL); Eyal Gilboa, Bat-Yam (IL); Tamas Koltai, Netanya (IL); Revital Lifshitz-Liron, Hertzlia (IL)

Correspondence Address: KENYON & KENYON LLP, One Broadway, New York, NY, 10004, US

(21) Appl. No.: 11/789,984

(22) Filed: Apr. 25, 2007

Related U.S. Application Data
(60) Provisional application No. 60/794,515, filed on Apr. 25, 2006.

(51) Int. Cl. A61K 31/66; A61P 19/00; C07F 9/22

(52) U.S. Cl. 514/102; 562/22

(57) ABSTRACT

The invention relates to solid crystalline forms of ibandronic acid, pharmaceutical formulations thereof, and methods of treatment therewith.

### CROSS-REFERENCE TO RELATED APPLICATIONS

This application claims the benefit of priority to U.S. provisional Application Ser. No. 60/794,515, filed Apr. 25, 2006, hereby incorporated by reference.

### FIELD OF THE INVENTION

The invention relates to the solid state chemistry of Ibandronic acid.

### BACKGROUND OF THE INVENTION

Ibandronate Sodium is a third-generation nitrogen-containing bisphosphonate characterized by an aliphatic tertiary amine side chain.

Ibandronate Sodium is a white crystalline powder. The free acid has a molecular weight of 319.23 (CAS No.: 114084-78-5). The monosodium salt (anhydrous) of the acid has a molecular weight of 341.23 (CAS No.: 138844-81-2). The monosodium salt monohydrate has a molecular weight of 359.23 (CAS No.: 138926-19-9).

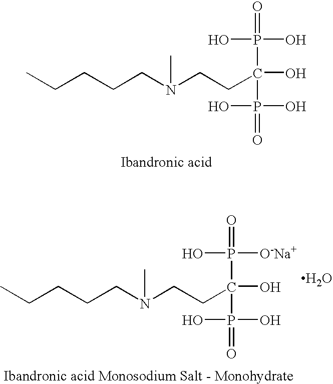

The preparation of ibandronic acid monosodium salt is described in, for example, U.S. Pat. No. 4,927,814 (“'814 patent”). The '814 patent describes the following reaction schemes:

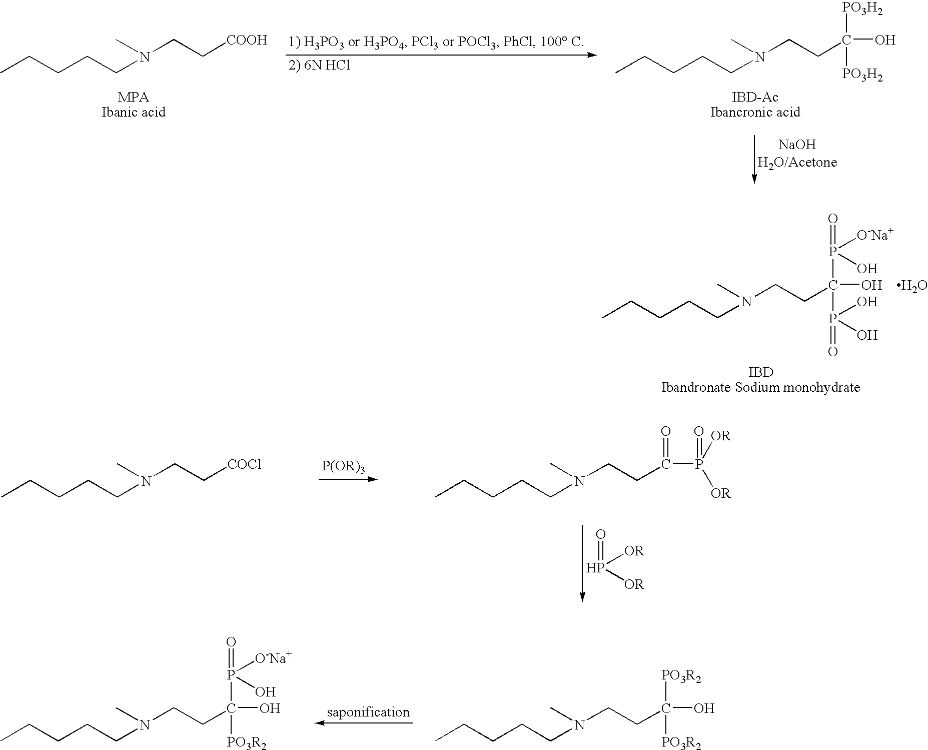

The preparation of a class of bisphosphonic acids, which includes ibandronic acid, is taught in U.S. Pat. No. 4,927,814 (“'814 patent”). In the process of the '814 patent, ion-exchange chromatography is used during work-up to isolate the bisphosphonic acid. See, e.g., '814 patent, col. 7, 11. 20-47 (example 1). The present inventors performed experiments based upon the procedures described in the '814 patent. See Examples 5-7 below. No solid material was obtained, but an oily precipitate was the crude product. The skilled artisan knows that solids are easier to manipulate than oils and consequently there is a need for a method of making a solid ibandronic acid.

Additional methods for the preparation of ibandronic acid are described in PCT publication No. WO 03/097655, in which ibandronic acid is obtained by reaction of a carboxylic acid, phosphorous acid and a halophosphorous compound in the presence of aromatic hydrocarbon or a silicone fluid.

The monosodium salt of ibandronic acid is marketed under the trade name BONIVA®. BONIVA® was developed by Hoffmann-La Roche for the treatment of bone disorders such as hypercalcaemia of malignancy, osteolysis, Paget's disease, osteoporosis and metastatic bone disease. BONIVA® is also marketed in Europe under the name BONDRONAT® for cancer-related bone complications. BONDRONAT® is available in ampoule with 1 ml concentrate for solution for infusion contains 1.125 mg of ibandronic acid monosodium salt monohydrate, corresponding to 1 mg of ibandronic acid.

Crystalline forms of ibandronic acid, as well as the amorphous form, are described in PCT publication No. WO 2006/002348.

Ibandronic acid can be used as an intermediate in the process for the preparation of Ibandronate sodium.

The invention relates to the solid state physical properties of ibandronic acid. These properties can be influenced by controlling the conditions under which ibandronic acid is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must use glidants such as colloidal silicon dioxide, talc, starch, or tribasic calcium phosphate.

Another important solid state property of a pharmaceutical compound is its rate of dissolution in aqueous fluid. The rate of dissolution of an active ingredient in a patient's stomach fluid can have therapeutic consequences since it imposes an upper limit on the rate at which an orally-administered active ingredient can reach the patient's bloodstream. The rate of dissolution is also a consideration in formulation syrups, elixirs, and other liquid medicaments. The solid state form of a compound can also affect its behavior on compaction and its storage stability.

These practical physical characteristics are influenced by the conformation and orientation of molecules in the unit cell, which define a particular polymorphic form of a substance. The polymorphic form can give rise to thermal behavior different from that of the amorphous material or another polymorphic form. Thermal behavior is measured in the laboratory by such techniques as capillary melting point, thermogravimetric analysis (TGA), and differential scanning calorimetry (DSC) and can be used to distinguish some polymorphic forms from others. A particular polymorphic form can also give rise to distinct spectroscopic properties that can be detectable by powder x-ray crystallography, solid state ¹³C NMR spectrometry, and infrared spectrometry.

Generally, the crystalline solid has improved chemical and physical stability over the amorphous form and forms with low crystallinity. The crystalline solid can also exhibit improved solubility, hygroscopicity, bulk properties, and/or flowability.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional polymorphic forms of ibandronic acid.

### SUMMARY OF THE INVENTION

In one embodiment, the invention encompasses a crystalline form of ibandronic acid (denominated “Form S15”) characterized by a powder x-ray diffraction pattern having peaks at about 8.2, 11.4, 11.8, 22.0 and 24.5±0.2 degrees two-theta.

In another embodiment, the invention encompasses a method for preparing the crystalline ibandronic acid Form S15 comprising: a) combining a halo-phosphorous compound and phosphorous acid with 3-N-methyl-N-pentylamino propionic acid or a salt thereof in a silicon oil to obtain a reaction mixture; b) heating the reaction mixture; c) combining the reaction mixture with water to obtain a biphasic mixture having an aqueous and a non-aqueous phase; d) separating the aqueous and non-aqueous phases; e) heating the aqueous phase; f) concentrating the aqueous phase to obtain a residue; g) adding about 40 to about 60 milliliters of ethanol per gram of the N-methyl-N-pentyl propionic acid or salt thereof to the residue to obtain a precipitate; and h) recovering the crystalline ibandronic acid Form S15 from the precipitate.

In another embodiment, the invention encompasses a method for preparing a pharmaceutically acceptable salt of ibandronic acid comprising: a) preparing crystalline ibandronic acid Form S15 by the above-described method; and b) converting the crystalline ibandronic acid Form S15 into a pharmaceutically acceptable salt of ibandronic acid.

In another embodiment, the invention encompasses a crystalline form of ibandronic acid (denominated “Form S16”) characterized by a powder x-ray diffraction pattern having peaks at about 4.7, 12.4, 16.4, 20.8 and 22.7±0.2 degrees two-theta.

In another embodiment, the invention encompasses a method for preparing crystalline ibandronic acid Form S16 comprising: a) combining a halo-phosphorous compound and phosphorous acid with 3-N-methyl-N-pentylamino propionic acid or a salt thereof in a silicon oil to obtain a reaction mixture; b) heating the reaction mixture; c) combining the reaction mixture with water to form a biphasic mixture having an aqueous and a non-aqueous phase; d) separating the aqueous and non-aqueous phases; e) heating the aqueous phase; f) concentrating the aqueous phase to obtain a residue; g) adding about 85 to about 100 milliliters of a C₂₋₄ alcohol per gram of the N-methyl-N-pentyl propionic acid or salt thereof to the residue to obtain a precipitate; and h) recovering the crystalline ibandronic acid Form S16 from the precipitate.

In another embodiment, the invention encompasses a method for preparing a pharmaceutically acceptable salt of ibandronic acid comprising: a) preparing crystalline ibandronic acid Form S16 by the above-described method; and b) converting the crystalline ibandronic acid Form S16 into a pharmaceutically acceptable salt of ibandronic acid.

In another embodiment, the invention encompasses crystalline ibandronic acid Form S15 or S16 having a maximum particle size of 500 μm. Preferably, the crystalline ibandronic acid Form S15 or S16 has a particle size of less than about 300 μm, more preferably less than about 200 μm, even more preferably less than about 100 μm, and most preferably less than about 50 μm.

### BRIEF DESCRIPTION OF THE FIGURES

FIG. 1 is a PXRD diffractogram of ibandronic acid Form S15 (obtained in Example 1).

FIG. 2 is a PXRD diffractogram of ibandronic acid Form S15 (obtained in Example 2).

FIG. 3 is a PXRD diffractogram of ibandronic acid Form S16 (obtained in Example 3).

FIG. 4 is a PXRD diffractogram of ibandronic acid Form S16 (obtained in Example 4).

### DETAILED DESCRIPTION OF THE INVENTION

The invention provides crystalline forms of ibandronic acid, as well methods of preparation of these crystalline forms. The invention further provides pharmaceutical compositions and methods for treating bone disorders.

As used herein, the term “room temperature” refers to a temperature of about 15° C. to about 30° C.

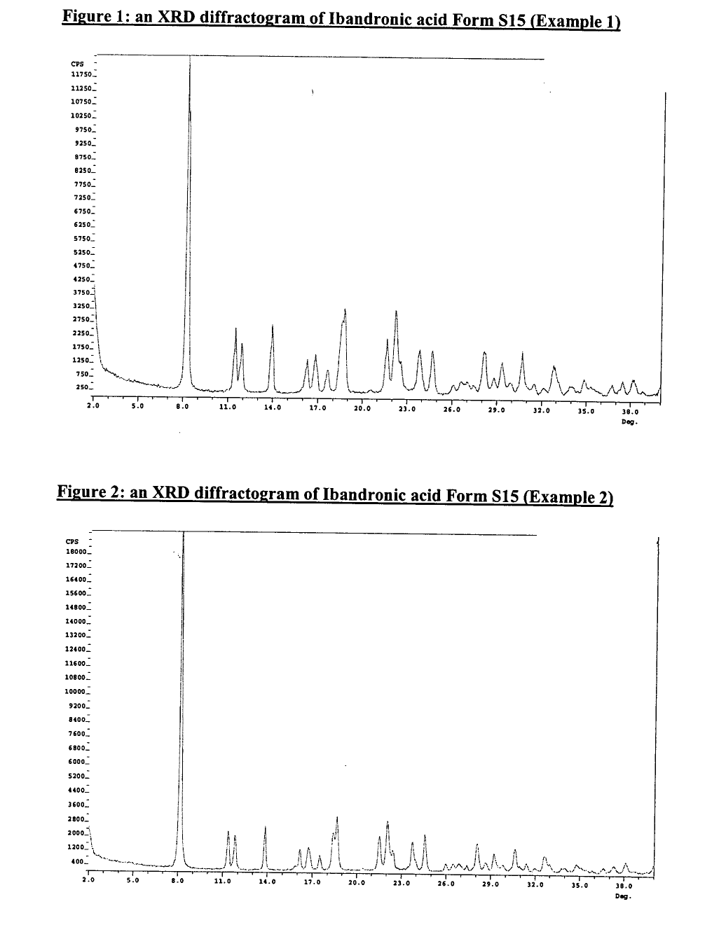

The invention encompasses a crystalline form of ibandronic acid, characterized by a powder x-ray diffraction (“PXRD”) pattern having peaks at about 8.2, 11.4, 11.8, 22.0 and 24.5±0.2 degrees two-theta (hereinafter referred to as “Form S15”). Form S15 can be further characterized by a PXRD pattern having peaks at about 13.8, 18.4, 18.7 and 21.5±0.2 degrees two-theta. Form S15 can be even further characterized by a PXRD pattern substantially as depicted in FIGS. 1 and 2. Typically, Form S15 does not contain more than about 5% by weight of ibandronic acid Form S16, based on the PXRD detection of the strongest characteristic peak of ibandronic acid Form S16, as defined below.

The invention further encompasses a method for preparing Form S15, comprising: a) combining a halo-phosphorous compound and phosphorous acid with 3-N-methyl-N-pentylamino propionic acid or a salt thereof in a silicon oil to obtain a reaction mixture; b) heating the reaction mixture; c) combining the reaction mixture with water to obtain a biphasic mixture having an aqueous and a non-aqueous phase; d) separating the aqueous and non-aqueous phases; e) heating the aqueous phase; f) concentrating the aqueous phase to obtain a residue; g) adding about 40 to about 60 milliliters (volumes) of ethanol per gram of the 3-N-methyl-N-pentylamino propionic acid or salt thereof to the residue to obtain a precipitate; and h) recovering Form S15 from the precipitate.

Preferably, the halo-phosphorous compound is selected from the group consisting of PCl₃, POCl₃, PBr₃, POBr₃, PCl₅, or PBr₅. More preferably, the halo-phosphorous compound is PCl₃.

Preferably, the salt of 3-N-methyl-N-pentylamino propionic acid is a hydrochloride or hydrobromide salt.

Suitable silicon oils (also known as silicone fluids) are miscible with organic solvents such as benzene, toluene, and carbon tetrachloride, but are insoluble in water. Preferred silicon oils include, but are not limited to, polydimethylsiloxane (“PDMS”), poly[oxy(dimethylsilene)], dimethicone, methylsilicone oil, Dow Coming® 200 fluid (a poly(dimethylsiloxane)), Wacker SWS101 fluid (a poly(dimethylsiloxane)), Baysilone® MPH 350 fluid, poly[oxy(methylphenylsilylene)], methylphenyl silicone oil, and Dow Coming® 710 fluid (phenyl methylsiloxane).

The halo-phosphorous compound may be added to the phosphorous acid and 3-N-methyl-N-pentylamino propionic acid or salt thereof slowly, in small aliquots, preferably dropwise. Alternatively, the halo-phosphorous compound may be added in one portion. The components of step a) are combined at about room temperature to about 78° C., preferably, about 73° C.

Typically, the reaction mixture in step b) is heated while stirring. Preferably, the reaction mixture in step b) is heated for about 3 to about 11 hours, more preferably for about 3 hours to about 9.5 hours, and most preferably for about 4 hours to about 8 hours. Preferably, the reaction mixture in step b) is heated at a temperature of about 60° C. to about 100° C., more preferably about 80° C. to about 90° C., and most preferably about 80° C. The water may be added to the reaction mixture slowly, in small aliquots, preferably dropwise. Preferably, the aqueous phase is heated at reflux temperature. The residue of step f) may be dissolved in water prior to the addition of the ethanol in step g). The ethanol of step g) may be added slowly, in small aliquots, preferably dropwise.

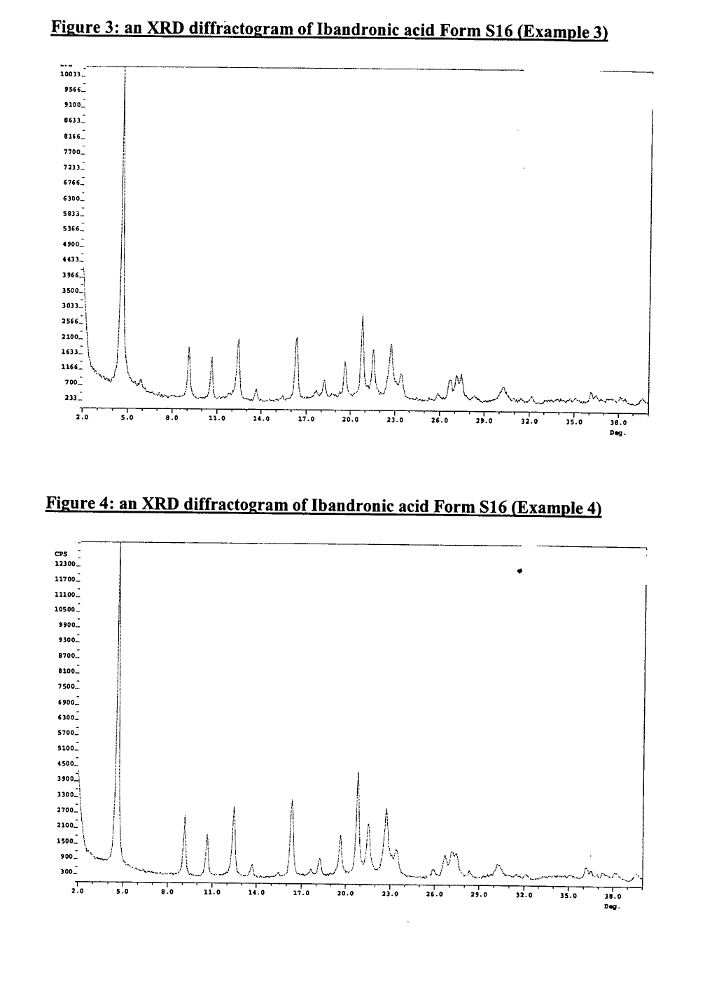

The invention further encompasses a crystalline form of ibandronic acid, characterized by a PXRD pattern having peaks at about 4.7, 12.4, 16.4, 20.8 and 22.7±0.2 degrees two-theta (hereinafter referred to as “Form S16”). Form S16 can be further characterized by a PXRD pattern having peaks at about 9.1, 10.6, 18.3, 19.6 and 21.6±0.2 degrees two-theta. Form S16 can be even further characterized by a PXRD pattern substantially as depicted in FIGS. 3 and 4. Typically, Form S16 does not contain more than about 5% by weight of ibandronic acid Form S10, based on the XRD detection of the strongest characteristic peak of ibandronic Form S10 (6.1±0.2 degrees two-theta). Ibandronic acid Form S10 is described in PCT publication No. WO 2006/002348, and is characterized by a PXRD pattern having peaks at about 4.8, 6.1, 12.0, 12.3, 16.4, 18.0 and 21.7±0.2 degrees two-theta.

The invention further encompasses a method for preparing ibandronic acid Form S16, comprising: a) combining a halo-phosphorous compound and phosphorous acid with 3-N-methyl-N-pentylamino propionic acid or a salt thereof in a silicon oil to obtain a reaction mixture; b) heating the reaction mixture; c) combining the reaction mixture with water to form a biphasic mixture having an aqueous and a non-aqueous phase; d) separating the aqueous and non-aqueous phases; e) heating the aqueous phase; f) concentrating the aqueous phase to obtain a residue; g) adding about 85 to about 100 milliliters (volumes) of a C₂₄ alcohol per gram of the N-methyl-N-pentyl propionic acid or salt thereof to the residue to obtain a precipitate; and h) recovering Form S16 from the precipitate.

Preferably, the halo-phosphorous compound is selected from the group consisting of PCl₃, POCl₃, PBr₃, POBr₃, PCl₅, or PBr₅. More preferably, the halo-phosphorous compound is PCl₃.

Preferably, the salt of 3-N-methyl-N-pentylamino propionic acid is the hydrochloride or hydrobromide salt.

The halo-phosphorous compound may be added to the phosphorous acid and 3-N-methyl-N-pentylamino propionic acid or salt thereof slowly, in small aliquots, preferably dropwise. Alternatively, the halo-phosphorous compound may be added as a single portion. The components of step a) may be combined at about room temperature, preferably about 25° C.

Typically, the reaction mixture in step b) is heated while stirring. Preferably, the reaction mixture in step b) is heated for about 3 to about 11 hours, more preferably for about 3 hours to about 9.5 hours, and most preferably for about 4 hours to about 8 hours. Preferably, the reaction mixture in step b) is heated at a temperature of about 60° C. to about 100° C., more preferably about 80° C. to about 90° C., and most preferably about 80° C. The water may be added to the reaction mixture slowly, in small aliquots, preferably dropwise. Preferably, the aqueous phase is heated at reflux temperature. The residue of step f) may be dissolved in water prior to the addition of the C₂₋₄ alcohol in step g). Preferably, the C₂₋₄ alcohol in step g) is selected from the group consisting of ethanol, 1-propanol and 2-propanol, where ethanol is most preferred. The reaction may include an additional step between steps c) and d) where 30% H₂O₂ is added to the two phases. The gradual addition of the 30% H₂O₂ results in improved phase separation.

The crystalline ibandronic acid forms S15 and S16 may be recovered by any means known in the art. For example, the crystalline form can be isolated by vacuum filtration. The processes can also include washing and/or drying the precipitated crystalline form. For example, the crystalline form can be washed with the same solvent used for dissolution. It can be dried in a vacuum oven at about 50° C. for about 24 hours or until constant weight, or it can be dried by evaporation.

The invention further encompasses crystalline ibandronic acid Form S15 or Form S16 having a maximum particle size of about 500μm. Typically, Form S15 or Form S16 has a particle size of less than about 300μm, preferably less than about 200μm, more preferably less than about 100 μm, and most preferably less than about 50 μm. Particle size is measured by at least one of the following methods: sieves, sedimentation, electrozone sensing (coulter counter), microscopy, and Low Angle Laser Light Scattering (LALLS).

The crystalline ibandronic acid Form S15 or S16 may subsequently be converted into a pharmaceutically acceptable salt of ibandronic acid by any method known to one of ordinary skill in the art. Preferably, the method comprises: preparing crystalline ibandronic acid Form S15 or Form S16 according to the above-described processes; and converting the crystalline ibandronic acid Form S15 or S16 into a pharmaceutically acceptable salt of ibandronic acid. Preferably, the pharmaceutically acceptable salt is a sodium salt.

The crystalline ibandronic acid Form S15 or S16, or pharmaceutically acceptable salts of ibandronic acid prepared the crystalline forms, may be formulated into pharmaceutical formulations with at least one pharmaceutically acceptable excipient.

Suitable pharmaceutically acceptable excipients include those known to one of ordinary skill in the art. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. AVICEL®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. EUDRAGIT®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. CARBOPOL®), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the invention include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the invention, the crystalline ibandronic acid or pharmaceutically acceptable salt thereof and any other solid excipients are suspended in a liquid carrier such as water, vegetable oil, alcohol, polyethylene glycol, propylene glycol, or glycerin, wherein the crystalline form of the ibandronic acid is maintained.

Liquid pharmaceutical compositions can contain emulsifying agents to disperse uniformly throughout the composition an active ingredient or other excipient that is not soluble in the liquid carrier. Emulsifying agents that can be useful in liquid compositions of the invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the invention, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the invention include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, the most preferred route of the invention is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the invention can be a capsule containing the composition, preferably a powdered or granulated solid composition of the invention, within either a hard or soft shell. The shell can be made from gelatin and optionally contain a plasticizer such as glycerin and sorbitol, and an opacifying agent or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the invention can comprise any of the aforementioned blends and granulates that were described with reference to tableting, but they are not subjected to a final tableting step.

The invention also provides methods of treating bone disorders comprising administering a pharmaceutical formulation of ibandronic acid or a pharamceutically acceptable salt thereof to a patient in need thereof. Bone disorders include, but are not limited to hypercalcaemia of malignancy, osteolysis, Paget's disease, osteoporosis and metastatic bone disease. Ibandronic acid or a pharmaceutically acceptable salt thereof is preferably formulated for administration by injection, preferably to a mammal, more preferably to a human. Ibandronic acid can be formulated, for example, as a viscous liquid suspension for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

BONIVA® and/or BONDRONAT® can be used as guidance for formulation. BONIVA® is available as an intravenous injection administered every 2-3 months and as an oral formulation. BONDRONAT® is available in ampoule with 1 ml concentrate for solution for infusion contains 1.125 mg of ibandronic monosodium salt monohydrate, corresponding to 1 mg of ibandronic acid.

Having thus described the invention with reference to particular preferred embodiments, other embodiments will become apparent to one skilled in the art from consideration of the specification. The invention is further defined by reference to the following examples describing in detail the synthesis of ibandronic acid Forms S15 and S16. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 can be used for guidance. It will be apparent to those skilled in the art that many modifications, both to materials and methods may be practiced without departing from the scope of the invention.

All references mentioned herein are incorporated in their entirety.

### EXAMPLES

Powder X-ray Diffraction

Powder X-ray diffraction analysis was performed using a SCINTAG powder X-ray diffiactometer model X'TRA equipped with a solid-state detector. Copper radiation of λ=1.5418 Å was used. The sample was introduced using a round standard aluminum sample holder with a round zero background quartz plate in the bottom. The scanning parameters were: range: 2-40 degrees two-theta; scan mode: continuous scan; step size: 0.05 deg.; and a rate of 5 deg./min.

High Performance Liquid Chromatography (“HPLC”)

Elution from the Amberlite column in Examples 5 to 7 was monitored by HPLC using a Hamilton type PRP-X100, Anion exchange, 250*4. 1 mm column at a temperature of 35° C. The eluent was a mixture containing 35% HNO₃, 45% KNO₃, and 20% ethanol. The flow rate was 2.0 mL/min and the detector was set at a wavelength of 240 nm. The injection volume of each sample was 50 μL and the diluent was water.

### Example 1

### Preparation of Ibandronic acid Form S15

A 500 ml reactor was loaded with silicon oil (210 ml), 3-N-methyl-N-pentylamino propionic acid hydrochloride (“ibanic acid hydrochloride” or “MPPA HCl”) (30 g) and H₃PO₃ (44 g) at room temperature. The mixture was heated to 73° C. and PCl₃ (47ml) was added drop-wise to form a reaction mixture over a period of 10 minutes. The reaction mixture was heated to 80° C. and stirred at 80° C. for 9.5 hours. Distilled water (210 ml) was then added drop-wise to form a biphasic mixture. The two phases were stirred for 0.5 hour. The lower aqueous phase was separated and hydrolyzed at reflux in a 250 ml reactor for 22 hours. Vacuum filtration through hyflo was done. The obtained solution was evaporated until dryness to obtain 64.3 g of colorless oil. The oily residue was dissolved in distilled water (10 ml) and absolute ethanol (1607 ml, ) was added drop-wise over a period of 25 minutes at room temperature. The slurry was stirred for 16 hours at room temperature and then it was cooled to 4° C. The product was isolated by vacuum filtration, washed with ethanol 96% (2×50 ml) and dried in a vacuum oven at 50° C. for 24 hours to obtain 22.53 g of ibandronic acid crystalline Form S15.

### Example 2

### Preparation of Ibandronic acid Form S15

A 500 ml reactor was loaded with silicon oil (210 ml), Ibanic acid hydrochloride (MPPA HCl) (30 g), H₃PO₃ (44 g) and PCl₃ (47 ml) at room temperature. The mixture was heated to 80° C. over a period of 2 hours. The reaction mixture was stirred for 3 hours. Distilled water (210 ml) was then added drop-wise to the reaction mixture to form a biphasic mixture. The two phases were stirred for 10 minutes. The lower aqueous phase was separated and hydrolyzed at reflux in a 250 ml reactor for 15 hours. The obtained solution was evaporated until dryness to obtain 75 g of colorless oil. Absolute ethanol (1440 ml) was added drop-wise over a period of 40 minutes at room temperature. The slurry was stirred for about 72 hours at room temperature. The product was isolated by vacuum filtration, washed with absolute ethanol (2×40 ml) and dried in a vacuum oven at 50° C. for 22 hours to obtain 23.5 g of ibandronic acid crystalline Form S15.

### Example 3

### Preparation of Ibandronic acid Form S16

A 500 ml reactor was loaded with silicon oil (105 ml), ibanic acid hydrochloride (MPPA HCl) (15 g) and H₃PO₃ (22 g) at room temperature. The mixture was heated to 80° C. in order to melt H₃PO₃. The mixture was then cooled to 25° C. and PCl₃ (23.4 ml) was added in one portion. The reaction mixture was heated to 80° C. over a period of 2 hours and stirred at 80° C. for 7.5 hours. Distilled water (105 ml) was then added drop-wise to the reaction mixture to form a biphasic mixture. The two phases were stirred for 10 minutes. The lower aqueous phase was separated and hydrolyzed at reflux in a 250 ml reactor during 15.5 hours. The obtained solution was evaporated until dryness to obtain 53.3 g of colorless oil. The oily residue was dissolved in distilled water (8 ml) and absolute ethanol (1333 ml) was added drop-wise over a period of 55 minutes at room temperature. The slurry was stirred for 16 hours at room temperature. The product was isolated by vacuum filtration, washed with absolute ethanol (2×25 ml) and dried in a vacuum oven at 50° C. during 20 hours to obtain 22 g of ibandronic acid crystalline Form S16.

### Example 4

### Preparation of Ibandronic acid Form S16

A 500 ml reactor was loaded with silicon oil (105 ml), Ibanic acid hydrochloride (MPPA HCl) (15 g), H₃PO₃ (22 g) and PCl₃ (19 ml) at room temperature. The reaction mixture was heated to 80° C. during 15 minutes and stirred at this temperature for 3 hours. Distilled water (105 ml) was added drop-wise to the reaction mixture to form a biphasic mixture. Then 30% H₂O₂ solution (3 ml) was added gradually to improve phase separation. The two phases were stirred for 30 minutes. The lower aqueous phase was separated and hydrolyzed at reflux in a 250 ml reactor during 18 hours. The obtained solution was evaporated until dryness to obtain 44.8 g of colorless oil. The oily residue was dissolved in distilled water (9 ml) and absolute ethanol (1500 ml) was added drop-wise during about 5 minutes at room temperature. The slurry was stirred for about 72 hours at room temperature. The product was isolated by vacuum filtration, washed with absolute ethanol (2×25 ml) and dried in a vacuum oven at 50° C. during 24 hours to obtain 20.2 g of ibandronic acid crystalline Form S16.

### Example 5

### Example Based Upon Example 9 of U.S. Pat. No. 4,927,814

15 g N-Methyl-N-pentylaminopropionic acid were kept for 23 hours at 100° C. with 8.8 g phosphorous acid and 18.7 ml phosphorous trichloride in 75 ml chlorobenzene. The solvent was then decanted off and the residue was stirred under reflux with 222 ml 6N HCl for 12.5 hours. Insoluble material was filtered off and the filtrate was concentrated and applied to column of Amberlite IR 120 (H+). The elution with water was monitored by HPLC, using the HPLC method described above. The desired fractions were combined, evaporated and stirred up with acetone to obtain a sticky oily precipitate as a crude product.

### Example 6

### Example Based Upon Example 9 of U.S. Pat. No. 4,927,814 (Substituting Methyl Ethyl Ketone for Acetone)

15 g N-Methyl-N-pentylaminopropionic acid were kept for 23 hours at 100° C. with 8.8 g phosphorous acid and 18.7 ml phosphorous trichloride in 75 ml chlorobenzene. The solvent was then decanted off and the residue was stirred under reflux with 222 ml 6N HCl for 12.5 hours. Insoluble material was filtered off and the filtrate was concentrated and applied to column of Amberlite IR 120 (H+). The elution with water was monitored by HPLC, using the HPLC method described above. The desired fractions were combined, evaporated and stirred up with methyl ethyl ketone (“MEK”) to obtain a sticky oily precipitate as a crude product.

### Example 7

### Example Based Upon Example 9 of U.S. Pat. No. 4,927,814 (Substituting Acetonitrile for Acetone)

15 g N-Methyl-N-pentylaminopropionic acid were kept for 23 hours at 100° C. with 8.8 g phosphorous acid and 18.7 ml phosphorous trichloride in 75 ml chlorobenzene. The solvent was then decanted off and the residue was stirred under reflux with 222 ml 6N HCl for 12.5 hours. Insoluble material was filtered off and the filtrate was concentrated and applied to column of Amberlite IR 120 (H+). The elution with water was monitored by HPLC, using the HPLC method described above. The desired fractions were combined, evaporated and stirred up with acetonitrile to obtain a sticky oily precipitate as a crude product.

We claim:

1. A crystalline form of ibandronic acid characterized by a powder x-ray diffraction pattern having peaks at about 8.2, 11.4, 11.8, 22.0 and 24.5±0.2 degrees two-theta.

2. The crystalline form of ibandronic acid of claim 1, further characterized by a powder x-ray diffraction pattern having peaks at about 13.8, 18.4, 18.7 and 21.5±0.2 degrees two-theta.

3. The crystalline form of ibandronic acid of claim 2, further characterized by a powder x-ray diffraction pattern substantially as depicted in FIG. 1 or FIG. 2.

4. A method for preparing the crystalline form of ibandronic acid of claim 1 comprising:
a) combining a halo-phosphorous compound and phosphorous acid with 3-N-methyl-N-pentylamino propionic acid or salt thereof in a silicon oil to obtain a reaction mixture;
b) heating the reaction mixture;
c) combining the reaction mixture with water to obtain a biphasic mixture having an aqueous and a non-aqueous phase;
d) separating the aqueous and non-aqueous phases;
e) heating the aqueous phase;
f) concentrating the aqueous phase to obtain a residue;
g) adding about 40 to about 60 milliliters of ethanol per gram of the N-methyl-N-pentyl propionic acid or salt thereof to the residue to obtain a precipitate; and
h) recovering the crystalline form of ibandronic acid of claim 1 from the precipitate.

5. The method of claim 4, wherein the salt of 3-N-methyl-N-pentylamino propionic acid is the hydrochloride salt or the hydrobromide salt.

6. The method of claim 4, wherein the halo-phosphorous compound is selected from the group consisting of PCl₃, POCl₃, PBr₃, POBr₃, PCl₅, or PBr₅.

7. The method of claim 4, wherein the halo-phosphorous compound is PCl₃.

8. The method of claim 4, wherein the halo-phosphorous compound is added dropwise to the phosphorous acid and 3-N-methyl-N-pentylamino propionic acid or salt thereof.

9. The method of claim 4, wherein the components of step a) are combined at a temperature of about room temperature to about 78° C.

10. The method of claim 4, wherein the reaction mixture in step b) is heated while stirring.

11. The method of claim 4, wherein the reaction mixture in step b) is heated for about 3 hours to about 11 hours.

12. The method of claim 4, wherein the reaction mixture in step b) is heated for about 3 hours to about 9.5 hours.

13. The method of claim 4, wherein the reaction mixture in step b) is heated for about 4 hours to about 8 hours.

14. The method of claim 4, wherein the reaction mixture in step b) is heated at a temperature of about 60° C. to about 100° C.

15. The method of claim 4, wherein the reaction mixture in step b) is heated at a temperature of about 80° C. to about 90° C.

16. The method of claim 4, wherein the reaction mixture is step b) is heated at a temperature of about 80° C.

17. The method of claim 4, wherein the aqueous phase is heated at reflux temperature.

18. The method of claim 4, wherein the residue of step f) is dissolved in water prior to the addition of ethanol.

19. A method for preparing a pharmaceutically acceptable salt of ibandronic acid comprising:
a) preparing a crystalline form of ibandronic acid by the method of claim 4; and
b) converting the crystalline form of ibandronic acid into a pharmaceutically acceptable salt of ibandronic acid.

20. The method of claim 19, wherein the pharmaceutically acceptable salt is a sodium salt.

21. A crystalline form of ibandronic acid characterized by a powder x-ray diffraction pattern having peaks at about 4.7, 12.4, 16.4, 20.8 and 22.7±0.2 degrees two-theta.

22. The crystalline form of ibandronic acid of claim 21, further characterized by a powder x-ray diffraction pattern having peaks at about 9.1, 10.6, 18.3, 19.6 and 21.6±0.2 degrees two-theta.

23. The crystalline form of ibandronic acid of claim 22, further characterized by a powder x-ray diffraction pattern substantially as depicted in FIG. 3 or FIG. 4.

24. A method for preparing the crystalline form of ibandronic acid of claim 21 comprising:
a) combining a halo-phosphorous compound and phosphorous acid with 3-N-methyl-N-pentylamino propionic acid or a salt thereof in a silicon oil to obtain a reaction mixture;
b) heating the reaction mixture;
c) combining the reaction mixture with water to form a biphasic mixture having an aqueous and a non-aqueous phase;
d) separating the aqueous and non-aqueous phases;
e) heating the aqueous phase;
f) concentrating the aqueous phase to obtain a residue;
g) adding about 85 to about 100 milliliters of a C₂₋₄ alcohol per gram of the N-methyl-N-pentyl propionic acid or salt thereof to the residue to obtain a precipitate; and
h) recovering the crystalline form of ibandronic acid of claim 21 from the precipitate.

25. The method of claim 24, wherein the salt of 3-N-methyl-N-pentylamino propionic acid is the hydrochloride salt or the hydrobromide salt.

26. The method of claim 24, wherein the halo-phosphorous compound is selected from the group consisting of PCl₃, POCl₃, PBr₃, POBr₃, PCl₅, or PBr₅.

27. The method of claim 24, wherein the halo-phosphorous compound is PCl₃.

28. The method of claim 24, wherein the halo-phosphorous compound is added dropwise to the phosphorous acid and 3-N-methyl-N-pentylamino propionic acid hydrochloride.

29. The method of claim 24, wherein the components of step a) are combined at a temperature of about room temperature.

30. The method of claim 24, wherein the reaction mixture in step b) is heated while stirring.

31. The method of claim 24, wherein the reaction mixture is step b) is heated for about 3 hours to about 11 hours.

32. The method of claim 24, wherein the reaction mixture in step b) is heated for about 3 hours to about 9.5 hours.

33. The method of claim 24, wherein the reaction mixture in step b) is heated for about 4 hours to about 8 hours.

34. The method of claim 24, wherein the reaction mixture in step b) is heated at a temperature of about 60° C. to about 100° C.

35. The method of claim 24, wherein the reaction mixture in step b) is heated at a temperature of about 80° C. to about 90° C.

36. The method of claim 24, wherein the reaction mixture of step b) is heated at a temperature of about 80° C.

37. The method of claim 24, wherein the aqueous phase is heated at reflux temperature.

38. The method of claim 24, wherein the C₂₋₄ alcohol is selected from the group consisting of ethanol, 1-propanol, and 2-propanol.

39. The method of claim 24, wherein the C₂₋₄ alcohol is ethanol.

40. The method of claim 24, further comprising adding 30% H₂O₂ to the two phases prior to the separation of step d).

41. The method of claim 24, wherein the residue of step f) is dissolved in water prior to the addition of the C₂₋₄ alcohol.

42. A method for preparing a pharmaceutically acceptable salt of ibandronic acid comprising:
a) preparing a crystalline form of ibandronic acid by the method of claim 24; and
b) converting the crystalline form of ibandronic acid into a pharmaceutically acceptable salt of ibandronic acid.

43. The method of claim 42, wherein the pharmaceutically acceptable salt is a sodium salt.

44. The crystalline form of ibandronic acid of claim 1, having a maximum particle size of about 500 μm.

45. The crystalline form of ibandronic acid of claim 44, having a particle size of less than about 300 μm.

46. The crystalline form of ibandronic acid of claim 44, having a particle size of less than about 200 μm.

47. The crystalline form of ibandronic acid of claim 44, having a particle size of less than about 100 μm.

48. The crystalline form of ibandronic acid of claim 44, having a particle size of less than about 50 μm.

49. The crystalline form of ibandronic acid of claim 21, having a maximum particle size of about 500 μm.

50. The crystalline form of ibandronic acid of claim 49, having a particle size of less than about 300 μm.

51. The crystalline form of ibandronic acid of claim 49, having a particle size of less than about 200 μm.

52. The crystalline form of ibandronic acid of claim 49, having a particle size of less than about 100 μm.

53. The crystalline form of ibandronic acid of claim 49, having a particle size of less than about 50 μm.
